SEQUENCE LISTING (11 sequences)

SEQ ID NO: 1 - DNA, 60 bp, Artificial Sequence
  source 1..60: /organism=“Artificial Sequence” /note=“SQfC_FWD” /mol_type=“unassigned DNA”
  misc_feature 9: /note=“C is 5-formylcytosine”
  misc_feature 32: /note=“C is 5-methylcytosine”
tacgatcacggcgaatccgatcgaatcgtttcggcgctttacgaagtgcgacagccttag

SEQ ID NO: 2 - DNA, 60 bp, Artificial Sequence
  source 1..60: /organism=“Artificial Sequence” /note=“SQfC_REV” /mol_type=“unassigned DNA”
  misc_feature 11: /note=“C is 5-formylcytosine”
  misc_feature 28: /note=“C is 5-hydroxymethylcytosine”
  misc_feature 33: /note=“C is 5-methylcytosine”
ctaaggctgtcgcacttcgtaaagcgccgaaacgattcgatcggattcgccgtgatcgta

SEQ ID NO: 3 - DNA, 100 bp, Artificial Sequence
  source 1..100: /organism=“Artificial Sequence” /note=“CEG_SC_1” /mol_type=“unassigned DNA”
  misc_feature 3,6,13: /note=“c is 5-methylcytosine”
ctcacccacaaccacaaacaatttaaatacgattaaataatattaatatattatcgattaaataataattaattaatatttgatgtgatgggtggtatgg

SEQ ID NO: 4 - DNA, 20 bp, Artificial Sequence
  source 1..20: /organism=“Artificial Sequence” /note=“CEG_Q3_Fwd” /mol_type=“unassigned DNA”
  misc_feature 3,6,13: /note=“c is 5-methylcytosine”
ctcacccacaaccacaaaca

SEQ ID NO: 5 - DNA, 20 bp, Artificial Sequence
  source 1..20: /organism=“Artificial Sequence” /note=“CEG_Q8_Rev” /mol_type=“unassigned DNA”
  misc_feature 6,10,16: /note=“c is 5-methylcytosine”
ccataccacccatcacatca

SEQ ID NO: 6 - DNA, 60 bp, Artificial Sequence; identical to SEQ ID NO: 1
  source 1..60: /organism=“Artificial Sequence” /note=“CEG_SQfC_Fwd” /mol_type=“unassigned DNA”
  misc_feature 9: /note=“c is 5-formylcytosine”
  misc_feature 41: /note=“c is 5-methylcytosine”

SEQ ID NO: 7 - DNA, 60 bp, Artificial Sequence; identical to SEQ ID NO: 2
  source 1..60: /organism=“Artificial Sequence” /note=“CEG_SQfC_Rev” /mol_type=“unassigned DNA”
  misc_feature 11: /note=“c is 5-formylcytosine”
  misc_feature 28,33: /note=“c is 5-methylcytosine”

SEQ ID NO: 8 - DNA, 24 bp, Artificial Sequence
  source 1..24: /organism=“Artificial Sequence” /note=“Oligo 9” /mol_type=“unassigned DNA”
  misc_feature 1,5,12,15,19: /note=“c is 5-methylcytosine”
caagcagaagacggcatacgagat

SEQ ID NO: 9 - DNA, 33 bp, Artificial Sequence
  source 1..33: /organism=“Artificial Sequence” /note=“Oligo 10” /mol_type=“unassigned DNA”
  misc_feature 2,4,6,10,11,12,15,17,20,22,24,27,28,32: /note=“c is 5-methylcytosine”
acactctttccctacacgacgctcttccgatct

SEQ ID NO: 10 - DNA, 58 bp, Artificial Sequence
  source 1..58: /organism=“Artificial Sequence” /note=“PCR_Uni_Fwd” /mol_type=“unassigned DNA”
aatgatacggcgaccaccgagatctacactctttccctacacgacgctcttccgatct

SEQ ID NO: 11 - DNA, 51 bp, Artificial Sequence
  source 1..51: /organism=“Artificial Sequence” /note=“PCR_IDX_Rev” /mol_type=“unassigned DNA”
  misc_feature 25..30: /note=“n is a or g or c or t/u, unknown, or other”
caagcagaagacggcatacgagatnnnnnngtgactggagttcagacgtgt

(19) United States
(12) Patent Application Publication
(10) Pub. No.: US 2017/0283870 A1
(43) Pub. Date: Oct. 5, 2017

(54) METHODS FOR DETECTION OF NUCLEOTIDE MODIFICATION

(71) Applicant: CAMBRIDGE EPIGENETIX LTD, Cambridge (GB)

(72) Inventors: Tobias William Barr OST, Cambridge (GB); Neil Matthew BELL, Cambridge (GB); Maria Chiara Erminia CATENAZZI, Cambridge (GB)

(21) Appl. No.: 15/508,520

(22) PCT Filed: Sep. 7, 2015
(86) PCT No.: PCT/GB2015/052582

§ 371 Date: Mar. 3, 2017

(30) Foreign Application Priority Data
Sep. 5, 2014 (GB) 1415761.4

(51) Int. Cl. C12Q 1/68

(52) U.S. Cl. CPC C12Q 1/6874; C12Q 1/6806; C12Q 1/6858; C12Q 2600/156

(57) ABSTRACT

This invention relates to improved methods and kits for identification of 5-formylcytosine (5fC) to be distinguished from cytosine (C) in a sample nucleotide sequence. Methods comprise reducing a first portion of polynucleotides which comprise the sample nucleotide sequence; treating the reduced first portion and a second portion of polynucleotides with bisulfite; sequencing the polynucleotides in the first and second portions of the population to produce first and second nucleotide sequences respectively and; identifying the residues in the first and second nucleotide sequences which correspond to a cytosine residue in the sample nucleotide sequence. These methods may be useful, for example in the analysis of genomic DNA and/or of RNA.

This invention relates to the detection of modified cytosine residues and, in particular, to the sequencing of nucleic acids that contain modified cytosine residues.

5-methylcytosine (5mC) is a well-studied epigenetic DNA mark that plays important roles in gene silencing and genome stability, and is found enriched at CpG dinucleotides (1). In metazoa, 5mC can be oxidised to 5-hydroxymethylcytosine (5hmC) by the ten-eleven translocation (TET) family of enzymes (2, 3). The overall levels of 5hmC are roughly 10-fold lower than those of 5mC and vary between tissues (4). Relatively high quantities of 5hmC (˜0.4% of all cytosines) are present in embryonic stem (ES) cells, where 5hmC has been suggested to have a role in the establishment and/or maintenance of pluripotency (2,3, 5-9). 5hmC has been proposed as an intermediate in active DNA demethylation, for example by deamination or via further oxidation of 5hmC to 5-formylcytosine (5fC) and 5-carboxycytosine (5cC) by the TET enzymes, followed by base excision repair involving thymine-DNA glycosylase (TDG) or failure to maintain the mark during replication (10). However, 5hmC may also constitute an epigenetic mark per se.

It is possible to detect and quantify the level of 5hmC present in total genomic DNA by analytical methods that include thin layer chromatography and tandem liquid chromatography-mass spectrometry (2, 11, 12). Mapping the genomic locations of 5hmC has thus far been achieved by enrichment methods that have employed chemistry or antibodies for 5hmC-specific precipitation of DNA fragments that are then sequenced (6-8, 13-15). These pull-down approaches have relatively poor resolution (10s to 100s of nucleotides) and give only relative quantitative information that is likely to be subject to distributional biasing during the enrichment. Quantifiable single nucleotide sequencing of 5mC has been performed using bisulfite sequencing (BS-Seq), which exploits the bisulfite-mediated deamination of cytosine to uracil for which the corresponding transformation of 5mC is much slower (16). However, it has been recognized that both 5mC and 5hmC are very slow to deaminate in the bisulfite reaction and so these two bases cannot be discriminated (17, 18). Two relatively new and elegant single molecule methods have shown promise in detecting 5mC and 5hmC at single nucleotide resolution. Single molecule real-time sequencing (SMRT) has been shown to detect derivatised 5hmC in genomic DNA (19). However, enrichment of DNA fragments containing 5hmC is required, which leads to loss of quantitative information (19). 5mC can be detected, albeit with lower accuracy, by SMRT (19). Furthermore, SMRT has a relatively high rate of sequencing errors (20), the peak calling of modifications is imprecise (19) and the platform has not yet sequenced a whole genome. Protein and solid-state nanopores can resolve 5mC from 5hmC and have the potential to sequence unamplified DNA molecules with further development (21, 22).

The present inventors have devised methods that allow modified cytosine residues, such as 5-methylcytosine (5mC), 5-hydroxymethylcytosine (5hmC) and 5-formylcytosine (5fC) to be distinguished from cytosine (C) at single nucleotide resolution. These methods are applicable to all sequencing platforms and may be useful, for example in the analysis of genomic DNA and/or of RNA.

Methods of oxidising and reducing cytosine bases are known (23-25). Methods of reduction described therein are unreliable, and rely on making solutions of unstable reagents immediately prior to use. Methods of the prior art involve either the addition of solid borohydride powder to aqueous DNA samples or the preparation of borohydride in water rather than alkaline solution immediately prior to use, rather than the provision of stable reagents suitable for use in reliable commercial kits.

An aspect of the invention provides a method of identifying a 5-formylcytosine residue in a sample nucleotide sequence comprising;
(i) providing a population of polynucleotides which comprise the sample nucleotide sequence,
(ii) reducing a first portion of said population by adding an alkaline borohydride solution, p1 (iii) treating the reduced first portion of said population and a second portion of said population with bisulfite,
(iv) sequencing the polynucleotides in the first and second portions of the population following steps ii) and iii) to produce first and second nucleotide sequences, respectively and;
(v) identifying the residues in the first and second nucleotide sequences which correspond to a 5-formylcytosine residue in the sample nucleotide sequence.

The population of polynucleotides may be single stranded prior to reduction. The reduction of single stranded rather than double stranded samples is more efficient, providing a higher efficiency of conversion, and requires a lower concentration of borohydride. The population of polynucleotides may be in an alkaline solution prior to exposure to borohydride, thereby ensuring the polynucleotides are single stranded.

The residues are identified in the first and second nucleotide sequences which correspond to a cytosine residue in the sample nucleotide sequence. Where the cytosine residues have been altered to uracil residues, the presence of unmodified cytosine bases is indicated. Where the cytosine residues have been prevented from being altered into uracil residues by the reducing step, the presence of 5-formylcytosine residues are indicated. The method is thus indicative of the presence of cytosine and formylcytosine residues and can distinguish between the two at each cytosine residue in the sample sequence.

For example, cytosine residues may be present at one or more positions in the sample nucleic acid sequence. The residues at these one or more positions in the first and second nucleotide sequences may be identified. A modified cytosine at a position in the sample nucleotide sequence may be identified from combination of residues identified in the first and second nucleotide sequences respectively (i.e. C and C, U and U, C and U, or U and C) at that position. The cytosine modifications which are indicated by different combinations are shown in table 10. In particular examples, unmodified C residues become U residues upon bisulfite treatment. 5-Formyl residues also become U residues upon bisulfite treatment. However if the formyl group is reduced to hydroxymethyl prior to bisulfite treatment, the base remains as C when treated with bisulfite. Thus the reduction step allows the differentiation of C and 5-formyl C which can not be distinguished by bisulfite treatment alone.

The methods described herein may be useful in identifying and/or distinguishing cytosine (C) and 5-formylcytosine (5fC) in a sample nucleotide sequence. For example, methods described herein may be useful in distinguishing one residue from the group consisting of cytosine (C), 5-methylcytosine (5mC), 5-hydroxymethylcytosine (5hmC) and 5-formylcytosine (5fC) from the other residues in the group.

Preferably, modified cytosine residues, such as 5-hydroxymethylcytosine, in the first portion of said population are not labelled, for example with substituent groups, such as glucose, before the oxidisation or reduction of step ii).

In some embodiments of the invention, a portion of polynucleotides from the population may be oxidised. For example, 5-hydroxymethylcytosine residues in the first portion of polynucleotides may be converted into 5-formylcytosine (5fC) by oxidation and the portion of polynucleotides then treated with bisulfite. The oxidation in addition to the reduction allows differentiation between methylcytosine and hydroxymethylcytosine.

A method of identifying a modified cytosine residue in a sample nucleotide sequence may comprise;
(i) providing a population of polynucleotides which comprise the sample nucleotide sequence,
(ii) reducing a first portion of said population by adding an alkaline borohydride solution,
(iii) oxidising a second portion of said population,
(iv) treating the reduced first portion, oxidised second portion and a third portion of said population with bisulfite,
(v) sequencing the polynucleotides in the first, second and third portions of the population following steps ii), iii) and iv) to produce first, second and third nucleotide sequences, respectively and;
(vi) identifying the residues in the first, second and third nucleotide sequences which correspond to a cytosine residue in the sample nucleotide sequence.

The population of polynucleotides may be single stranded prior to reduction. The reduction of single stranded rather than double stranded samples is more efficient, providing a higher efficiency of conversion, and requires a lower concentration of borohydride. The population of polynucleotides may be in an alkaline solution prior to exposure to borohydride, thereby ensuring the polynucleotides are single stranded.

The identification of a residue at a position in all of the first, second and third nucleotide portions as cytosine is indicative that the cytosine residue in the sample nucleotide sequence is 5-methylcytosine. 5-Methylcytosine is not affected by the reduction or oxidation steps.

The identification of a residue at a position in all of the first, second and third nucleotide portions as uracil is indicative that the cytosine residue in the sample nucleotide sequence is unmodified cytosine. Unmodified cytosine is not affected by the reduction or oxidation steps.

The identification of a residue which in the first and third portions is cytosine, and in the second portion is uracil is indicative that the cytosine residue in the sample nucleotide sequence is 5-hydroxymethylcytosine. The hydroxymethyl group is unchanged by reduction, and remains as C upon bisulfite treatment, whereas it becomes oxidised to formyl C, which becomes uracil upon bisulfite treatment.

The identification of a residue which in the first portion is cytosine, and in the second and third portions is uracil is indicative that the cytosine residue in the sample nucleotide sequence is 5-formylcytosine. The formyl group is unchanged by oxidation, and becomes uracil upon bisulfite treatment, whereas it becomes reduced to hydroxymethylcytosine, which remains as cytosine upon bisulfite treatment.

Thus the four states C, 5mC, 5hmC and 5fC can be distinguished by comparing the same locations across the separate sequencing reactions on the different portions of the sample.

The first, second and/or third portions of the polynucleotide population may be treated with bisulfite and/or sequenced simultaneously or sequentially. The reducing step does not have to be performed prior to the oxidation step. The method indicated by roman numerals merely shows that the reduction and optional oxidation steps have to be carried out separately, not chronologically.

In some embodiments in which the first portion is reduced in step ii), oxidation treatment of the second portion may not be required to identity or distinguish a modified cytosine residue in the sample nucleotide sequence. For example, Table 10 shows that reduction and bisulfite treatment of the first portion of the polynucleotide population is sufficient to identify 5-formylcytosine in the sample nucleotide sequence. A method of identifying 5-formylcytosine in a sample nucleotide sequence or distinguishing 5-formylcytosine from cytosine (C), 5-methylcytosine (5mC) and 5-hydroxymethylcytosine (5hmC) in a sample nucleotide sequence may comprise;
(i) providing a population of polynucleotides which comprise the sample nucleotide sequence,
(ii) reducing said population by adding an alkaline borohydride solution,
(iii) treating the reduced population with bisulfite,
(iv) sequencing the polynucleotides in the population following steps ii) and iii) to produce a treated nucleotide sequence, and;
(v) identifying a uracil residue in the treated nucleotide sequence which corresponds to a cytosine residue in the sample nucleotide sequence, wherein the presence of a uracil in the treated nucleotide sequence is indicative that the cytosine residue in the sample nucleotide sequence is 5-formylcytosine (5fC).

In order to differentiate between 5mC and 5hmC, the optional oxidation step may be introduced. A summary of the cytosine modifications at a position in the sample nucleotide sequence which are indicated by specific combinations of cytosine and uracil at the position in the first, second and third nucleotide sequences is shown in Table 10. The four structures C, 5mC, 5hmC and 5fC are shown in Table 11.

The sample nucleotide sequence may be already known or it may be determined. The sample nucleotide sequence is the sequence of untreated polynucleotides in the population i.e. polynucleotides which have not been oxidised, reduced or bisulfite treated. In the sample nucleotide sequence, modified cytosines are not distinguished from cytosine. 5-Methylcytosine, 5-formylcytosine and 5-hydroxymethylcytosine are all indicated to be or identified as cytosine residues in the sample nucleotide sequence. For example, any of the methods described herein may further comprise;
providing a fourth portion of the population of polynucleotides comprising sample nucleotide sequence; and,
sequencing the polynucleotides in the fourth portion to produce the sample nucleotide sequence.

The sequence of the polynucleotides in the fourth portion may be determined by any appropriate sequencing technique.

The positions of one or more cytosine residues in the sample nucleotide sequence may be determined. This may be done by standard sequence analysis. Since modified cytosines are not distinguished from cytosine, cytosine residues in the sample nucleotide sequence may be cytosine, 5-methylcytosine, 5-formylcytosine or 5-hydroxymethylcytosine.

The first and second nucleotide sequences and, optionally the third nucleotide sequence, may be compared to the sample nucleotide sequence. For example, the residues at positions in the first and second sequences and, optionally the third nucleotide sequence, corresponding to the one or more cytosine residues in the sample nucleotide sequence may be identified.

The modification of a cytosine residue in the sample nucleotide sequence may be determined from the identity of the nucleotides at the corresponding positions in the first and second nucleotide sequences and, optionally the third nucleotide sequence.

The polynucleotides in the population all contain the same sample nucleotide sequence i.e. the sample nucleotide sequence is identical in all of the polynucleotides in the population.

The effect of different treatments on cytosine residues within the sample nucleotide sequence can then be determined, as described herein.

The sample nucleotide sequence may be a genomic sequence. For example, the sequence may comprise all or part of the sequence of a gene, including exons, introns or upstream or downstream regulatory elements, or the sequence may comprise genomic sequence that is not associated with a gene. In some embodiments, the sample nucleotide sequence may comprise one or more CpG islands.

The sample polynucleotides may be in single stranded or double stranded form. If the polynucleotides are in single stranded form, the concentration of borohydride may be lower that the concentration required for double stranded polynucleotides. The sample polynucleotides may therefore be denatured before the borohydride is added. The denaturation may take the form of heat or alkali. The method may include the step of treating the sample with alkali before the borohydride is added.

The final concentration of borohydride used to reduce the nucleic acid sample may be in the range of 10 to 500 mM. The concentration may be less than 0.2 M. Prior art reduction conditions carried out on double stranded DNA use solid borohydride added to the solution at a higher concentration than 0.2 M. The final concentration of borohydride may be 10 to 200 mM. The final concentration of borohydride may be 20 to 200 mM.

Suitable polynucleotides include DNA, preferably genomic DNA, and/or RNA, such as genomic RNA (e.g. mammalian, plant or viral genomic RNA), mRNA, tRNA, rRNA and non-coding RNA.

The polynucleotides comprising the sample nucleotide sequence may be obtained or isolated from a sample of cells, for example, mammalian cells, preferably human cells.

Suitable samples include isolated cells and tissue samples, such as biopsies.

Modified cytosine residues including 5hmC and 5fC have been detected in a range of cell types including embryonic stem cells (ESCS) and neural cells (2, 3, 11, 37, 38).

Suitable cells include somatic and germ-line cells.

Suitable cells may be at any stage of development, including fully or partially differentiated cells or non-differentiated or pluripotent cells, including stem cells, such as adult or somatic stem cells, foetal stem cells or embryonic stem cells.

Suitable cells also include induced pluripotent stem cells (iPSCs), which may be derived from any type of somatic cell in accordance with standard techniques.

For example, polynucleotides comprising the sample nucleotide sequence may be obtained or isolated from neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes.

Suitable cells include disease-associated cells, for example cancer cells, such as carcinoma, sarcoma, lymphoma, blastoma or germ line tumour cells.

Suitable cells include cells with the genotype of a genetic disorder such as Huntington's disease, cystic fibrosis, sickle cell disease, phenylketonuria, Down syndrome or Marfan syndrome.

Methods of extracting and isolating genomic DNA and RNA from samples of cells are well-known in the art. For example, genomic DNA or RNA may be isolated using any convenient isolation technique, such as phenol/chloroform extraction and alcohol precipitation, caesium chloride density gradient centrifugation, solid-phase anion-exchange chromatography and silica gel-based techniques.

In some embodiments, whole genomic DNA and/or RNA isolated from cells may be used directly as a population of polynucleotides as described herein after isolation. In other embodiments, the isolated genomic DNA and/or RNA may be subjected to further preparation steps.

The genomic DNA and/or RNA may be fragmented, for example by sonication, shearing or endonuclease digestion, to produce genomic DNA fragments. A fraction of the genomic DNA and/or RNA may be used as described herein. Suitable fractions of genomic DNA and/or RNA may be based on size or other criteria. In some embodiments, a fraction of genomic DNA and/or RNA fragments which is enriched for CpG islands (CGIs) may be used as described herein.

The genomic DNA and/or RNA may be denatured, for example by heating or treatment with a denaturing agent. Suitable methods for the denaturation of genomic DNA and RNA are well known in the art.

In some embodiments, the genomic DNA and/or RNA may be adapted for sequencing before oxidation or reduction and bisulfite treatment, or bisulfite treatment alone. The nature of the adaptations depends on the sequencing method that is to be employed. For example, for some sequencing methods, primers may be ligated to the free ends of the genomic DNA and/or RNA fragments following fragmentation. Suitable primers may contain 5mC to prevent the primer sequences from altering during oxidation or reduction and bisulfite treatment, or bisulfite treatment alone, as described herein. In other embodiments, the genomic DNA and/or RNA may be adapted for sequencing after oxidation, reduction and/or bisulfite treatment, as described herein.

Following fractionation, denaturation, adaptation and/or other preparation steps, the genomic DNA and/or RNA may be purified by any convenient technique.

Following preparation, the population of polynucleotides may be provided in a suitable form for further treatment as described herein. For example, the population of polynucleotides may be in aqueous solution in the absence of buffers before treatment as described herein.

Polynucleotides for use as described herein may be single or double-stranded.

The population of polynucleotides may be divided into two, three, four or more separate portions, each of which contains polynucleotides comprising the sample nucleotide sequence. These portions may be independently treated and sequenced as described herein.

Preferably, the portions of polynucleotides are not treated to add labels or substituent groups, such as glucose, to 5-hydroxymethylcytosine residues in the sample nucleotide sequence before oxidation and/or reduction.

The first portion of the population of polynucleotides comprising the sample nucleotide sequence may be reduced.

Reduction converts any 5-formylcytosine in the sample nucleotide sequence to 5-hydroxymethylcytosine. The reduction may be carried out by adding an alkaline borohydride solution.

The use of a stabilised solution of borohydride allows improved kits for better control of the amount of borohydride added to the reaction. Borohydride solutions can be stabilised by a high pH. Thus the use of alkaline solution of borohydride gives control over the amount of active borohydride added to the nucleic acid sample. Prior art conditions of making borohydride solutions immediately prior to use means that the amount of active borohydride in solution depends of the purity and source of the borohydride, the level of decomposition of the solid prior to making the solution, the composition and pH of the buffer used to make the solution, and how long the solution is kept before use.

The need to make up a solution from a reactive powder immediately prior to use does not allow the reducing agent to be supplied in a reliable way. One improvement described herein is therefore improved method and kits whereby a stabilised borohydride solution is provided, thus allowing commercial distribution of improved kits, and more reliable methods whereby the reducing conditions can be controlled and reliably repeated.

The alkaline borohydride solution can be a metal borohydride. The borohydride can be lithium, sodium or potassium. The borohydride can be NaBH₄. Suitable reducing agents include NaBH₄, NaCNBH₄ and LiBH₄.

The alkaline borohydride can be supplied at a pH greater than 10.0. The solution can be sodium borohydride at pH greater than 10.0.

The alkaline conditions can be provide by a solution containing hydroxide. The hydroxide can be lithium, sodium or potassium. The hydroxide can be present at a concentration of greater than 5 Moles/L. The hydroxide can be present at a concentration of greater than 10 Moles/L.

The optional oxidising agent is any agent suitable for generating an aldehyde from an alcohol. The oxidising agent or the conditions employed in the oxidation step may be selected so that any 5-hydroxymethylcytosine is selectively oxidised. Thus, substantially no other functionality in the polynucleotide is oxidised in the oxidation step. The oxidising step therefore does not result in the reaction of any thymine or 5-methylcytosine residues, where such are present. The agent or conditions are selected to minimise or prevent any degradation of the polynucleotide.

The use of an oxidising agent may result in the formation of some corresponding 5-carboxycytosine product. The formation of this product does not negatively impact on the methods of identification described herein. Under the bisulfite reaction conditions that are used to convert 5-formylcytosine to uracil, 5-carboxycytosine is observed to convert to uracil also. It is understood that a reference to 5-formylcytosine that is obtained by oxidation of 5-hydroxymethylcytosine may be a reference to a product also comprising 5-carboxycytosine that is also obtained by that oxidization.

The oxidising agent may be a non-enzymatic oxidising agent, for example, an organic or inorganic chemical compound.

Suitable oxidising agents are well known in the art and include metal oxides, such as KRuO₄, MnO₂ and KMnO₄. Particularly useful oxidising agents are those that may be used in aqueous conditions, as such are most convenient for the handling of the polynucleotide. However, oxidising agents that are suitable for use in organic solvents may also be employed where practicable.

In some embodiments, the oxidising agent may comprise a perruthenate anion (RuO₄⁻). Suitable perruthenate oxidising agents include organic and inorganic perruthenate salts, such as potassium perruthenate (KRuC4) and other metal perruthenates; tetraalkylammonium perruthenates, such as tetrapropylammonium perruthenate (TPAP) and tetrabutylammonium perruthenate (TBAP); polymer-supported perruthenate (PSP) and tetraphenylphosphonium ruthenate.

Advantageously, the reducing and/or oxidising agent or the reducing conditions may also preserve the polynucleotide in a denatured state.

Following treatment with the reducing agent, the polynucleotides in the first portion may be purified.

Purification may be performed using any convenient nucleic acid purification technique. Suitable nucleic acid purification techniques include spin-column chromatography.

The polynucleotide may be subjected to further, repeat reducing steps. Such steps are undertaken to maximise the conversion of 5-formylcytosine to 5-hydroxymethylcytosine. This may be necessary where a polynucleotide has sufficient secondary structure that is capable of re-annealing. Any annealed portions of the polynucleotide may limit or prevent access of the reducing agent to that portion of the structure, which has the effect of protecting 5-formylcytosine from reduction.

In some embodiments, the first portion of the population of polynucleotides may for example be subjected to multiple cycles of treatment with the reducing agent followed by purification. For example, one, two, three or more than three cycles may be performed.

Following oxidation and reduction, the portions of the population are treated with bisulfite. A portion of the population which has not been oxidised or reduced is also treated with bisulfite. Bisulfite treatment converts both cytosine and 5-formylcytosine residues in a polynucleotide into uracil. A portion of the population may be treated with bisulfite by incubation with bisulfite ions (HSO₃²⁻).

The use of bisulfite ions (HSO₃²⁻) to convert unmethylated cytosines in nucleic acids into uracil is standard in the art and suitable reagents and conditions are well known to the skilled person (39-42). Numerous suitable protocols and reagents are also commercially available (for example, EpiTect™, Qiagen NL; EZ DNA Methylation™ Zymo Research Corp CA; CpGenome Turbo Bisulfite Modification Kit; Millipore).

A feature of the methods described herein is the conversion of unmethylated cytosine to uracil. This reaction is typically achieved through the use of bisulfite. However, in general aspects of the invention, any reagent or reaction conditions may be used to effect the conversion of cytosine to uracil. Such reagents and conditions are selected such that little or no 5-methylcytosine reacts, and more specifically such that little or no 5-methylcytosine reacts to form uracil. The reagent, or optionally a further reagent, may also effect the conversion of 5-formylcytosine or 5-carboxycytosine to cytosine or uracil.

Following the incubation, the portions of polynucleotides may be immobilised, washed, desulfonated, eluted and/or otherwise treated as required.

In some embodiments, the first, second and third portions of polynucleotides from the population may be amplified following treatment as described above. This may facilitate further manipulation and/or sequencing. Sequence alterations in the first, second and third portions of polynucleotides are preserved following the amplification. Suitable polynucleotide amplification techniques are well known in the art and include PCR. The presence of a uracil (U) residue at a position in the first, second and/or third portions of polynucleotide may be indicated or identified by the presence of a thymine (T) residue at that position in the corresponding amplified polynucleotide.

As described above, polynucleotides may be adapted after oxidation, reduction and/or bisulfite treatment to be compatible with a sequencing technique or platform. The nature of the adaptation will depend on the sequencing technique or platform. For example, for Solexa-Illumina sequencing, the treated polynucleotides may be fragmented, for example by sonication or restriction endonuclease treatment, the free ends of the polynucleotides repaired as required, and primers ligated onto the ends.

Polynucleotides may be sequenced using any convenient low or high throughput sequencing technique or platform, including Sanger sequencing (43), Solexa-Illumina sequencing (44), Ligation-based sequencing (SOLiD™) (45), pyrosequencing (46); strobe sequencing (SMRT™) (47, 48); and semiconductor array sequencing (Ion Torrent™) (49).

Suitable protocols, reagents and apparatus for polynucleotide sequencing are well known in the art and are available commercially.

The residues at positions in the first, second and/or third nucleotide sequences which correspond to cytosine in the sample nucleotide sequence may be identified.

The modification of a cytosine residue at a position in the sample nucleotide sequence may be determined from the identity of the residues at the corresponding positions in the first, second and, optionally, third nucleotide sequences, as described above.

The extent or amount of cytosine modification in the sample nucleotide sequence may be determined. For example, the proportion or amount of 5-hydroxymethylcytosine and/or 5-methylcytosine in the sample nucleotide sequence compared to unmodified cytosine may be determined.

Polynucleotides as described herein, for example the population of polynucleotides or 1, 2, 3, or all 4 of the first, second, third and fourth portions of the population, may be immobilised on a solid support.

A solid support is an insoluble, non-gelatinous body which presents a surface on which the polynucleotides can be immobilised. Examples of suitable supports include glass slides, microwells, membranes, or microbeads. The support may be in particulate or solid form, including for example a plate, a test tube, bead, a ball, filter, fabric, polymer or a membrane. Polynucleotides may, for example, be fixed to an inert polymer, a 96-well plate, other device, apparatus or material which is used in a nucleic acid sequencing or other investigative context. The immobilisation of polynucleotides to the surface of solid supports is well-known in the art. In some embodiments, the solid support itself may be immobilised. For example, microbeads may be immobilised on a second solid surface.

In some embodiments, the first, second, third and/or fourth portions of the population of polynucleotides may be amplified before sequencing. Preferably, the portions of polynucleotide are amplified following the treatment with bisulfite.

Suitable methods for the amplification of polynucleotides are well known in the art.

Following amplification, the amplified portions of the population of polynucleotides may be sequenced.

Nucleotide sequences may be compared and the residues at positions in the first, second and/or third nucleotide sequences which correspond to cytosine in the sample nucleotide sequence may be identified, using computer-based sequence analysis.

Nucleotide sequences, such as CpG islands, with cytosine modification greater than a threshold value may be identified. For example, one or more nucleotide sequences in which greater than 1%, greater than 2%, greater than 3%, greater than 4% or greater than 5% of cytosines are hydroxymethylated may be identified.

Computer-based sequence analysis may be performed using any convenient computer system and software. A typical computer system comprises a central processing unit (CPU), input means, output means and data storage means (such as RAM). A monitor or other image display is preferably provided. The computer system may be operably linked to a DNA and/or RNA sequencer.

For example, a computer system may comprise a processor adapted to identify modified cytosines in a sample nucleotide sequence by comparison with first, second and/or third nucleotide sequences as described herein. For example the processor may be adapted;
(a) identify the positions of cytosine residues in the sample nucleotide sequence,
(b) identify the residues in the first, second and/or third nucleotide sequences at the positions of cytosine residues in the sample nucleotide sequence,
(c) determine from the identities of said residues the presence or absence of modification of the cytosine residue at the positions in the sample nucleotide sequence.

The sample nucleotide sequence and the first second and third nucleotide sequences may be entered into the processor automatically from the DNA and/or RNA sequencer. The sequences may be displayed, for example on a monitor.

The computer system may further comprise a memory device for storing data. Nucleotide sequences such as genomic sequences, and the positions of 5fC, 5hmC and other modified cytosine residues may be stored on another or the same memory device, and/or may be sent to an output device or displayed on a monitor. This may facilitate the mapping of modified cytosines, such as 5hmC and 5fC, in genomic DNA.

The identification and mapping of cytosine modifications, such as 5fC and 5hmC, in the genome may be useful in the study of neural development and function, and cell differentiation, division and proliferation, as well as the prognosis and diagnosis of diseases, such as cancer.

The identification and/or mapping of modified cytosines such as 5fC and 5hmC, using the methods described herein may therefore be useful in disease.

Another aspect of the invention provides a kit for use in a method of identifying a modified cytosine residue in a sample nucleotide sequence as described above, comprising;
a stabilised reducing agent; and,
a bisulfite reagent.

Suitable reducing agents and bisulfite reagents are described above.

The kit may comprise a kit for use in a method of identifying a 5-formylcytosine residue comprising;
(i) an alkaline borohydride solution; and,
(ii) a bisulfite reagent.

The kit may further contain an alkaline solution. The alkaline solution may be used to ensure the nucleic acid is single stranded prior to addition of the borohydride solution.

The alkaline borohydride solution can be a metal borohydride. The borohydride can be lithium, sodium or potassium. The borohydride can be NaBH₄. Suitable reducing agents include NaBH₄, NaCNBH₄ and LiBH₄.

The alkaline borohydride or alkaline solution can be supplied at a pH greater than 10.0. The alkaline borohydride or alkaline solution can be supplied at a pH greater than 14.0. The solution can be sodium borohydride at pH greater than 10.0. The solution can be sodium borohydride at pH greater than 14.0. The borohydride can be present in the range of 1-30 weight % of the solution. The borohydride can be present in the range of 10-20 weight % of the solution.

The alkaline conditions can be provided by a solution containing hydroxide. The hydroxide can be lithium, sodium or potassium. The hydroxide can be present at a concentration of greater than 1 Moles/L. The hydroxide can be present at a concentration of greater than 5 Moles/L. The hydroxide can be present at a concentration of greater than 10 Moles/L.

A kit may further comprise a population of control polynucleotides comprising one or more modified cytosine residues, for example cytosine (C), 5-methylcytosine (5mC), 5-hydroxymethylcytosine (5hmC) or 5-formylcytosine (5fC). In some embodiments, the population of control polynucleotides may be divided into one or more portions, each portion comprising a different modified cytosine residue.

The kit may include instructions for use in a method of identifying a modified cytosine residue as described above.

A kit may include one or more other reagents required for the method, such as buffer solutions, sequencing and other reagents. A kit for use in identifying modified cytosines may include one or more articles and/or reagents for performance of the method, such as means for providing the test sample itself, including DNA and/or RNA isolation and purification reagents, and sample handling containers (such components generally being sterile).

Various further aspects and embodiments of the present invention will be apparent to those skilled in the art in view of the present disclosure.

All documents mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments that are described.

Certain aspects and embodiments of the invention will now be illustrated by way of example and with reference to the figures described below.

Table 10 shows sequencing outcomes for cytosine and modified cytosines subjected to various treatments.

Table 11 shows the structures of cytosine (1a), 5-methylcytosine (5mC; 1b), 5-hydroxymethylcytosine (5hmC; 1c) and 5-formylcytosine (5fC; 1d)

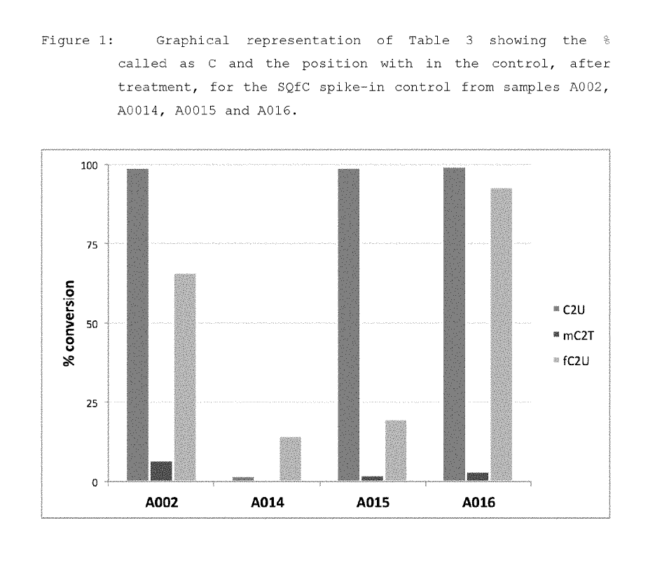

FIG. 1 shows a graphical representation of Table 3 showing the % called as C and the position with in the control, after treatment, for the SQfC spike-in control from samples A002, A0014, A0015 and A016.

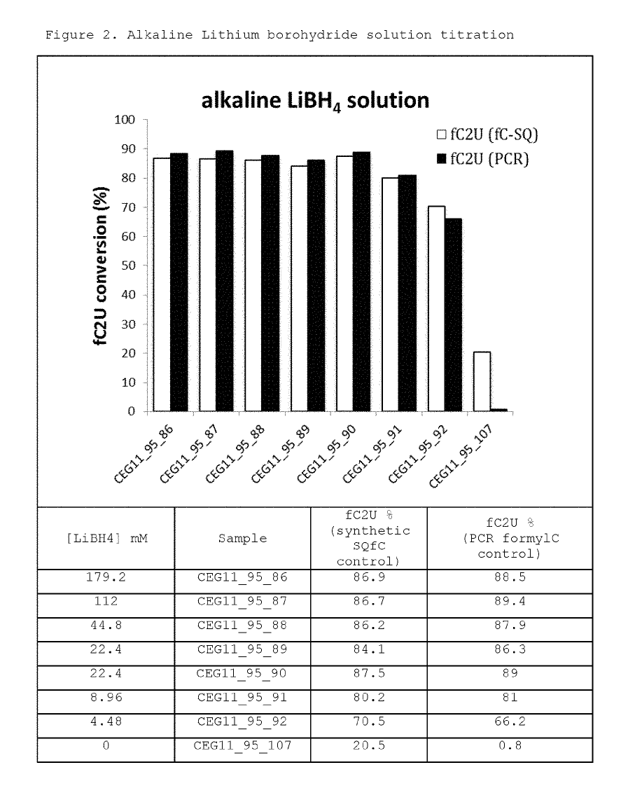

FIG. 2 shows the conversion rates from the sequencing data obtained from a library spiked in with 1.5 0 of PCR formylC control and 1.5% synthetic SQfC control for different lithium borohydride concentrations.

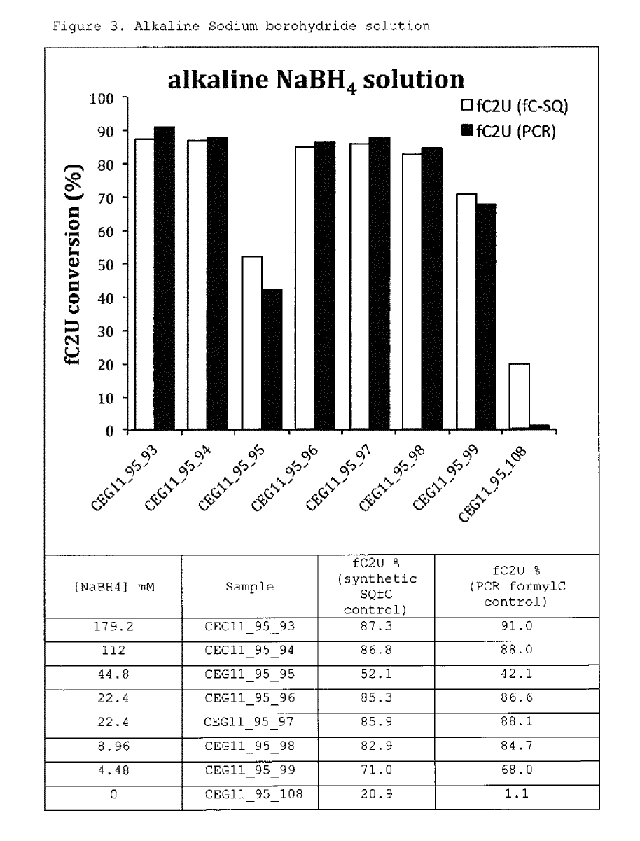

FIG. 3 shows the sequencing data for the titration of sodium borohydride solutions.

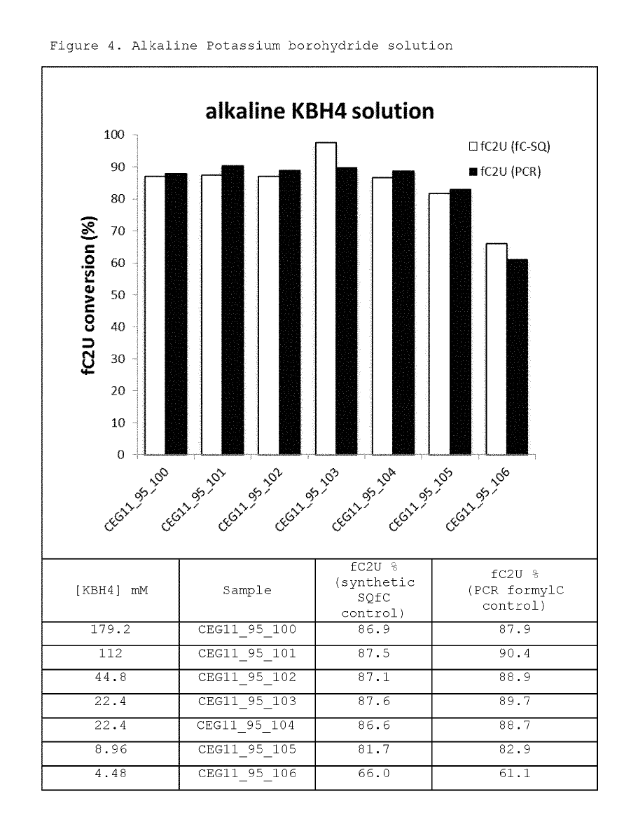

FIG. 4 shows the conversion data for the titration of potassium borohydride solutions. Comparable levels of fC2U conversion are observed with all alkaline borohydride solutions tested at [BH4-]>20 mM, regardless of the nature of the cationic counter-ion.

Table 9 shows the conversion rates from the sequencing data obtained from a library spiked in with 2.5 0 of synthetic formylC control and 10% PCR formylC control for 8 repeats using sodium borohydride solution and 2 replicates with no reductant.

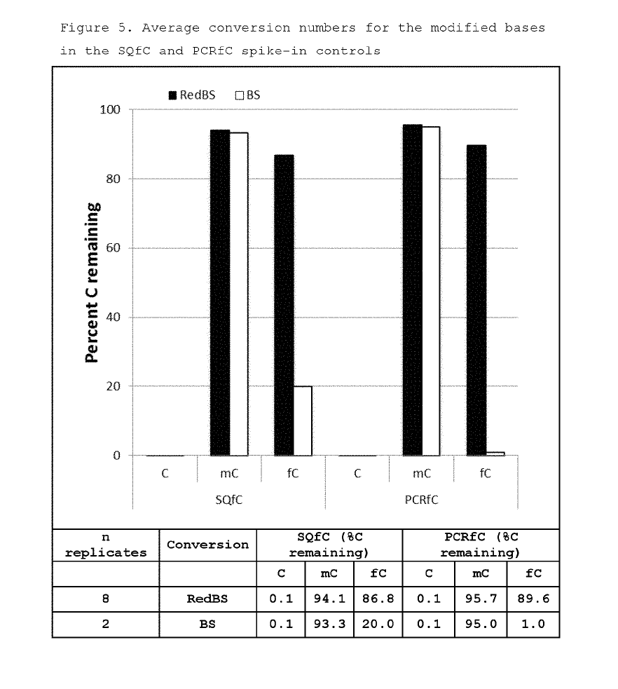

FIG. 5 shows the average of the reproducibility.

### EXPERIMENTAL PROTOCOL

Reagents

Spike-in Sequencing Controls (SQfC)

Modified oligonucleotides were prepared by ATDbio using standard solid phase oligo synthesis and phosphoramidite chemistry.

SQfC_FWD:

5′pTACGATCAXGGCGAATCCGATCGAATCGTTTZGGCGCTTTACGAAGTGCGACAGCCTTAG

SQfC REV:

5′pCTAAGGCTGTXGCACTTCGTAAAGCGC5GAAAZGATTCGATCGGATTCGCCGTGATCGTA

X=5-formylcytosine

Z=5-methylcytosine

Reagents
Illumina TruSeq LT DNA Sample Prep Kit (P/N: FC-121-2001)
CEGX TrueMethyl Oxidative Bisulfite Kit
Sodium borohydride solution ˜12% w/v in 14 M NaOH (Sigma 452904)

Equipment
Diagenode Bioruptor DNA sonication device
Illumina MiSeq DNA sequencer
BsExpress Pipeline (https://code.google.com/p/oxbs-sequencing-qc/wiki/bsExpressDoc)

Experiment

Preparation of the SQfC spike-in Sequencing Control.

Equal amounts of the SQfC_FWD and SQfC_REV controls were diluted together in 10 mM Tris-HCl (pH 8.0), Incubated at 90°C. for 2 mins, before the oligos were cooled to 25° C. over 1 hour to allow hybridization of the SQfC controls. The annealed duplex (SQfC) control was then diluted to 1.5 ng/μL.

Preparation of DNA Library.

To 1 μg of sonicated Lambda DNA (100-1000 bp) was added to 0.3% w/w (3 ng, 2 μL of the 1.5 ng/μL stock) of the SQfC control duplex and the double stranded DNA was prepared for sequencing using standard Illumina TruSeq LT DNA Sample Prep kit, according to the manufacturers standard protocol. A total of three libraries were made (see below) with indexes A014, A015 & A016.

DNA Denaturation.

To 23 μL of library prepared DNA containing a 0.3% w/w spiked-in SQfC control duplex was added 1 μL 1.0 M NaOH. The reagents were vortexed briefly, Centrifuged, and incubated for 30 minutes at 37° C. to ensure full denaturation.

Conversion of the Denatured TruSeq Lambda Libraries.

The denatured lambda TruSeq libraries containing 0.3% w/w SQfC control duplex were subjected to a variety of conversions as outlined in the Table 1 below. Specific conversion reactions are detailed in the text below.

TABLE 1
The individual treatments for A002, A014, A015 and
A016.

| Index | Reductant | Oxidant | Bisulfite |
|---|---|---|---|
| A002 | — | Mock | Yes |
| A014 | Mock | — | Mock |
| A015 | Yes | — | Yes |
| A016 | — | Yes | Yes |

Mock Oxidation of A002: To 24 uL of the denatured DNA A016 was added 1 μL of 50 mM NaOH and incubate at 40° C. for 30.

Mock Reduction of A014: To 24 μL of the denatured DNA A014 was added 1 μL of 14 M sodium hydroxide solution and incubate at RT for 60 minutes in the dark. Final concentration of hydroxide=0.56M.

Reduction of A015: To 24 uL of the denatured DNA A015 was added 1 μL of the sodium borohydride reduction solution (Sodium borohydride solution ˜12 wt. % in 14 M NaOH)and incubate at RT for 60 minutes in the dark. Final concentration of borohydride=0.17M and hydroxide=0.56M in RedBS reaction.

Oxidation of A016: To 24 uL of the denatured DNA A016 was added 1 μL CEGX oxidation solution was added and the reaction was held on ice for 1 hour, with occasional vortexing) and incubate at 40° C. for 30.

Bisulfite Conversions: To the 25 μL of A002, A014, A015 and A016 DNA was added 175 uL of CEGX Bisulfite Reagent. The reagents were mixed by vortexing briefly and centrifuged.

Sample A014 was immediately worked-up using the CEGX TrueMethyl post-bisulfite purification protocol, while samples A002, A015 and A016 were incubated as described within the CEGX User Guide prior to the post-bisulfite purification protocol.

All samples were PCR amplified and purified as described in the CEGX TrueMethyl protocol.

Sequencing of the Converted TruSeq Lambda Libraries.

Samples A002, A014, A015 and A016 were pooled into an equimolar mix following conversion and the pool was sequenced on an Illumina MiSeq sequencer (75+6 cycle SBS run, V2.0 MiSeq SBS chemistry P/N: MS-102-2001). Fastq files for each sample (A002,

A014, A015 and A016) were automatically generated following completion of sequencing and basecalling (MCS v2.3.0.8).

Results

The fastq files for indexes A002, A014, A015 and A016 were analyzed using the standard BsExpress pipeline, the results are summarized below in Table 3 and FIG. 1.

TABLE 3
Cytosine conversion percentages for the SQfC
spike-in control from samples A002, A0014, A0015 and
A016.

| | | % mC2T | % fC2U |
|---|---|---|---|
| Sample | % C2U conversion | conversion | conversion |
| A002 | 98.92 | 6.76 | 66.04 |
| A014 | 1.65 | 0.51 | 14.34 |
| A015 | 98.97 | 2.20 | 19.96 |
| A016 | 99.34 | 3.17 | 93.01 |

Conversion KEY:
C2U = cytosine to uracil;
mC2T = 5-methylcytosine to thymine;
fC2U = 5-formylcytosine to uracil

Conclusions

Conversion of A002: This corresponds to bisulfite-only conditions. Observations as expected, high C2U conversion rate; low mC2T conversion rate and intermediate fC2U rate (as a result of a proportion of the fC residues within the oligo being in the hydrate form—a byproduct of the solid phase oligo synthesis process).

Conversion of A014: This corresponds to mock redBS conditions and represents the NaOH treated background. Observations as expected, low C2U, mC2T and fC2U conversions. All consistent with no exposure to bisulfite. Expect all modified cytosines to read as C post conversion.

Conversion of A015: This corresponds to RedBS conditions. Observations as expected, high C2U conversion and low mC2T conversion rates. In this reaction, fC is converted to 5-hydroxymethylcytosine by treatment with the reductant solution and the resulting 5-hmC is resistant to bisulfite conversion. A high proportion of fC read as cytosine following RedBS treatment (low fC2U conversion).

Conversion of A016: This corresponds to oxBS conditions. Observatons as expected, high C2U conversion and low mC2T conversion rates. After treatment with oxidant solution, fC2U conversion is very high (presumably due to oxidation of 5-formyl cytosine to 5-carboxycytosine and the facile bisulfite conversion of 5-caC to uracil).

Alternative Alkaline Borohydride Solutions

Materials:

TABLE 4
Oligonucleotide sequences

| | | Internal |
|---|---|---|
| Name | Sequence (5′ → 3′) | mods: |
| CEG_SC_1 | CT5AC5CACAAC5ACAAACAATTT | 5 = 5 mC |
| | AAATACGATTAAATAATATTAATA | |
| | TATTATCGATTAAATAATAATTAA | |
| | TTAATATTTGATGTGATGGGTGGT | |
| | ATGG | |
| CEG_Q3_Fwd | CT5AC5CACAAC5ACAAACA | 5 = 5 mC |
| CEG_Q8_Rev | CCATA5CAC5CATCA5ATCA | 5 = 5 mC |
| CEG_SQfC_Fwd | pTACGATCA3GGCGAATCCGATCG | 3 = 5 fC |
| | AATCGTTT5GGCGCTTTACGAAGT | 5 = 5 mC |
| | GCGACAGCCTTAG | |
| CEG_SQfC_Rev | pCTAAGGCTGT3GCACTTCGTAAA | 3 = 5 fC |
| | GCGC5GAAA5GATTCGATCGGATT | 5 = 5 mC |
| | CGCCGTGATCGTA | |
| Oligo 9 | 5AAG5AGAAGA5GG5ATA5GAGAT | 5 = mC |
| Oligo 10 | A5A5T5TTT555TA5A5GA5G5T5 | 5 = mC |
| | TT55GAT5T | |
| PCR_Uni_Fwd | AATGATACGGCGACCACCGAGATC | |
| | TACACTCTTTCCCTACACGACGCT | |
| | CTTCCGATCT | |
| PCR_IDX_Rev | CAAGCAGAAGACGGCATACGAGAT | |
| | NNNNNNGTGACTGGAGTTCAGACG | |
| | TGT | |

TABLE 5
Reagents used

| Reagent Name | Catalogue number | From |
|---|---|---|
| Lithium borohydride | 222356 | Sigma-Aldrich |
| Sodium borohydride | 71321 | Sigma-Aldrich |
| Potassium borohydride | 438472 | Sigma-Aldrich |
| Lithium hydroxide | 545856 | Sigma-Aldrich |
| Sodium hydroxide | S8045 | Sigma-Aldrich |
| Potassium hydroxide | P5958 | Sigma-Aldrich |
| 12% Sodium borohydride | 452904 | Sigma-Aldrich |
| solution in 14M NaOH | | |

TABLE 6
Illumina N6 index sequences

| Index # | Sequence |
|---|---|
| 1 | ATCACG |
| 2 | CGATGT |
| 3 | TTAGGC |
| 4 | TGACCA |
| 5 | ACAGTG |
| 6 | GCCAAT |
| 7 | CAGATC |
| 8 | ACTTGA |
| 9 | GATCAG |
| 10 | TAGCTT |
| 11 | GGCTAC |
| 12 | CTTGTA |
| 13 | AGTCAA |
| 14 | AGTTCC |
| 15 | ATGTCA |
| 16 | CCGTCC |
| 18 | GTCCGC |
| 19 | GTGAAA |
| 20 | GTGGCC |
| 21 | GTTTCG |
| 22 | CGTACG |
| 23 | GAGTGG |
| 25 | ACTGAT |
| 27 | ATTCCT |

Step 1: Preparation of Alkaline Borohydride Solutions

Sodium borohydride, potassium borohydride and lithium borohydride solutions were prepared by dissolving each borohydride salt in its corresponding 1 M hydroxide solution (ie, 1 M sodium hydroxide, 1 M potassium hydroxide or 1 M lithium hydroxide).

All borohydride solutions were made to a 0.56 M stock, from which titrations of 280, 112 and 56 mM were made by dilution of the original borohydride stock in the corresponding 1M hydroxide solution.

Step 2: Preparation of the PCR FormylC Control (CEG_SC_1)

The PCR formylC control was designed to contain 2 formylC in its sequence and also a recognition site for Taq^{α}I. FormylC was introduced into the sequence during PCR, and reactions were set up by adding 2 1 Template (CEG_SC_1) at 1 ng/μL, 5 DreamTaq Buffer 10× (NEB), 4 μL primer CEG_Q3_Fwd, 4 μL CEG_Q8_Rev, 2 μL 10 mM dATP, 2 μL 10 mM dGTP, 2 μL 10 mM dTTP, 2 μL 10 mM formyl dCTP, 0.25 μL DreamTaq (5U/μL) (NEB) and 26.75 μL ultra pure water.

Thermocycling conditions consisted of an initial denaturation step at 95° C. for 2 minutes, followed by 35 cycles of:

Denaturation at 95° C. for 30 seconds

Annealing at 57° C. for 30 seconds

Extension at 72° C. for 15 seconds

The products obtained were purified with 2× 30% PEG Ampure XP Beads (30% PEG-10000, 1 M NaCl, 1 mM EDTA, 10 mM Tris pH 8) according to manufacturers instructions but using 80:20 freshly prepared acetonitrile:water instead of 80:20 ethanol:water. Samples were eluted from the beads in 17 μL ultra pure water. Samples were then quantified by Qubit HS dsDNA assay kit.

Step 3: Preparation of the Synthetic formylC Control (CEG_SQfC)

The synthetic formylC control was prepared by hybridizing CEG_SQfC_Fwd (100 μM stock) and CEG_SQfC_Rev (100 μM stock) in 1× Anneal buffer (10 mM Tris pH 7.4 and 10 mM NaCl). The oligomers were annealed by heating to 97.5° C. for 2 minutes 30 seconds, then were cooled to 40° C. by decreasing the temperature of 0.1° C. per second, and were then held at 40° C. for 15 minutes.

After library preparation, the DNA was then quantified by Qubit HS dsDNA assay kit.

Step 4: Library Preparation

The sheared yeast genomic DNA (250 bp, 5 μg) was spiked in with 1.5% PCR formylC control and 1.5% synthetic SQfC control. Libraries were prepared using the NEBNext DNA Library Prep Master Mix Set for Illumina (NEB) following the manufacturer's specifications, and using 10 μL of the methylated adapter pair (25 μM) during the adapter ligation step.

The methylated adapter pair was prepared by annealing Oligo 9 (100 μM stock) and Oligo 10 (100 μM stock) to a final concentration of 25 μM each in 1× Anneal buffer (10 mM Tris pH 7.4 and 10 mM NaCl). The oligomers were hybridized by heating to 95° C. for 3 minutes, then were cooled to 14° C. by decreasing the temperature of 0.1° C. per second.

After library preparation, the DNA was then quantified by Qubit HS dsDNA assay kit.

Step 5: Denaturing Step

400 ng of the yeast DNA spiked with 1.5% PCR formyiC control and 1.5% synthetic SQfC control from step 4 in a total volume of 9.5 μL in ultra pure water was denatured in 0.5 μL of either lithium or sodium or potassium hydroxide 1 M solution at 37° C. for 30 minutes.

Step 6: Reduction

A titration of the reductant solution was added to the denatured DNA from step 5. The reductant solutions of LiBH₄, NaBH₄ or KBH₄ were used at final concentrations of 179.2, 112, 44.8, 22.4, 8.96 and 4.48 mM as illustrated in Table 7, to a final volume of 25 μL. One reaction was reduced with 1 μL of the 12% NaBH₄ in 14 M NaOH solution from Sigma-Aldrich. Each reaction was incubated at room temperature in the dark for 60 minutes.

TABLE 7
Reaction conditions
DNA denatured in LiOH 1M and reduced with LiBH₄

| | | Denatured | Reductant | LiOH | | Final |
|---|---|---|---|---|---|---|
| | Index (6N) | DNA from | Vol (μL) | 1M Vol | H₂O Vol | [Reductant] |
| Sample ID | (step 8) | step 5 (μL) | [stock] | (μL) | (μL) | [mM] |
| CEG11_95_86 | 1 | 10 | 8 [560 mM] | | 7 | 179.2 |
| CEG11_95_87 | 2 | 10 | 5 [560 mM] | | 10 | 112 |
| CEG11_95_88 | 3 | 10 | 2 [560 mM] | | 13 | 44.8 |
| CEG11_95_89 | 4 | 10 | 2 [280 mM] | | 13 | 22.4 |
| CEG11_95_90 | 5 | 10 | 2 [280 mM] | 6 | 7 | 22.4 |
| CEG11_95_91 | 6 | 10 | 2 [112 mM] | | 13 | 8.96 |
| CEG11_95_92 | 7 | 10 | 2 [56 mM] | | 13 | 4.48 |
| CEG11_95_107 | 23 | 10 | — | 2 | 13 | 0 |

DNA denatured in NaOH 1M and reduced with NaBH₄

| | | Denatured | Reductant | NaOH | | Final |
|---|---|---|---|---|---|---|
| | Index (6N) | DNA from | Vol (μL) | 1M Vol | H₂O Vol | [Reductant] |
| Sample ID | (step 8) | step 5 (μL) | [stock] | (μL) | (μL) | [mM] |
| CEG11_95_93 | 8 | 10 | 8 [560 mM] | | 7 | 179.2 |
| CEG11_95_94 | 9 | 10 | 5 [560 mM] | | 10 | 112 |
| CEG11_95_95 | 10 | 10 | 2 [560 mM] | | 13 | 44.8 |
| CEG11_95_96 | 11 | 10 | 2 [280 mM] | | 13 | 22.4 |
| CEG11_95_97 | 12 | 10 | 2 [280 mM] | 6 | 7 | 22.4 |
| CEG11_95_98 | 13 | 10 | 2 [112 mM] | | 13 | 8.96 |
| CEG11_95_99 | 14 | 10 | 2 [56 mM] | | 13 | 4.48 |
| CEG11_95_108 | 25 | 10 | — | 2 | 13 | 0 |

DNA denatured in KOH 1M and reduced with KBH₄

| | | Denatured | Reductant | KOH | | Final |
|---|---|---|---|---|---|---|
| | Index (6N) | DNA from | Vol (μL) | 1M Vol | H₂O Vol | [Reductant] |
| Sample ID | (step 8) | step 5 (μL) | [stock] | (μL) | (μL) | [mM] |
| CEG11_95_100 | 15 | 10 | 8 [560 mM] | | 7 | 179.2 |
| CEG11_95_101 | 16 | 10 | 5 [560 mM] | | 10 | 112 |
| CEG11_95_102 | 18 | 10 | 2 [560 mM] | | 13 | 44.8 |
| CEG11_95_103 | 19 | 10 | 2 [280 mM] | | 13 | 22.4 |
| CEG11_95_104 | 20 | 10 | 2 [280 mM] | 6 | 7 | 22.4 |
| CEG11_95_105 | 21 | 10 | 2 [112 mM] | | 13 | 8.96 |
| CEG11_95_106 | 22 | 10 | 2 [56 mM] | | 13 | 4.48 |
| CEG11_95_109 | 25 | 10 | — | 2 | 13 | 0 |
| CEG11_95_110 | 27 | 10 | 1 [12% NaBH₄ | | 14 | 126.88 |
| | | | in 14M NaOH] | | | |

Step 7: Bisulfite Conversion

The yeast genomic DNA with controls that underwent reduction was bisulfite converted using the TrueMethyl conversion kit (CEGX) following the manufacturers specification. The DNA was then quantified by Qubit ssDNA assay kit.

Step 8: PCR Amplification

PCR amplification was performed on an Agilent Surecycler 8800 thermocycler using a quarter (˜6 μL) of the bisulfite converted DNA and 1 U of VeraSeq Ultra DNA polymerase (Enzymatics). Thermocycling conditions consisted of an initial denaturation step at 95° C. for 2 minutes, followed by 15 cycles of:

Denaturation at 95° C. for 30 sec

Annealing at 60° C. for 30 sec

Extension at 72° C. for 1 min 30 sec

And a final extension step at 72° C. for 5 min

The primers that were used are PCR_Uni_Fwd and PCR_IDX_Rev (Table 4). The latter primer includes a sequence that hybridizes to an Illumina flow cell and contains a specific index tag (represented by a string of 6N nucleotides) (Table 6). PCR products were purified as described in step 7.

The products obtained were purified with 2× 18% PEG Ampure XP Beads (18% PEG-8000, 1 M NaCl, 1 mM EDTA, 10 mM Tris pH 8) according to manufacturers instructions but using 80:20 freshly prepared acetonitrile:water instead of 80:20 ethanol:water. Samples were eluted from the beads in 15 μl ultra pure water. Both controls were then checked on a bioanalyzer and quantified by Qubit HS dsDNA assay kit and by qPCR using the Illumina library Quantification kit (KAPA Biosystems).

Step 9. Sequencing and Analysis:

Sequencing was carried out on an Illumina Miseq sequencer with a paired end run (R1 110 bp and R2 40 bp long). The 25 libraries were pooled at 2 nM and then diluted to 20 pM before loading on to the flow cell and sequenced, according to the manufacturers instructions. The raw output fastq read sequences were quality filtered and trimmed to remove the adapter sequences with the software Trim Galore. The data was aligned to the PCR formylC control and to the synthetic SQfC control with Bismark software and visualized by SeqMonk.

FIG. 2 shows the conversion rates from the sequencing data obtained from a library spiked in with 1.5% of PCR formylC control and 1.5% synthetic SQfC control for different lithium borohydride concentrations. FIG. 3 shows the sequencing data for the titration of sodium borohydride solutions. FIG. 4 shows the conversion data for the titration of potassium borohydride solutions. CEG11_95_109 was not sequenced, due to shortage of indexing primers (ie, same indexing primer was used for both samples CEG11_95_108 and CEG11_95_109).

CEG11_95_110 fC2U conversion rate for the synthetic control was 86.3%, whereas fC2U conversion rate for PCR formylC control was 88.3%.

Conclusion:

Comparable levels of fC2U conversion are observed with all alkaline borohydride solutions tested at [BH4-]>20 mM, regardless of the nature of the cationic counter-ion.

### EXAMPLE

### Repeatability Test using Alkaline Borohydride Solution

Step 1: Borohydride New Formulation

12% Sodium borohydride solution in 14 M NaOH from Sigma-Aldrich (Catalogue number: 452904) was diluted 2.5× in ultra pure water to a final concentration of 1.27 M NaBH₄ and 5.6 M NaOH, resulting in a working solution of 50 mM sodium borohydride.

Step 2: Preparation of the PCR FormylC Control (CEG_SC_1)

The PCR formylC control was designed to contain 2 formylC in its sequence and also a recognition site for Taq°I. FormylC was introduced into the sequence during PCR, and reactions were set up by adding 2 1 Template (CEG_SC_1) at 1 ng/μL, 5 DreamTaq Buffer 10× (NEB), 4 μL primer CEG_Q3_Fwd, 4 μL CEG_Q8_Rev, 2 μL 10 mM dATP, 2 μL 10 mM dGTP, 2 μL 10 mM dTTP, 2 μL 10 mM formyl dCTP, 0.25 μL DreamTaq (5U/μL) (NEB) and 26.75 μL ultra pure water.

Thermocycling conditions consisted of an initial denaturation step at 95° C. for 2 minutes, followed by 35 cycles of:

Denaturation at 95° C. for 30 seconds

Annealing at 57° C. for 30 seconds

Extension at 72° C. for 15 seconds

The products obtained were purified with 2× 30% PEG Ampure XP Beads (30% PEG-10000, 1 M NaCl, 1 mM EDTA, 10 mM Tris pH 8) according to manufacturers instructions but using 80:20 freshly prepared acetonitrile:water instead of 80:20 ethanol:water. Samples were eluted from the beads in 17 μL ultra pure water. Samples were then quantified by Qubit HS dsDNA assay kit.

Step 3: Preparation of the Synthetic FormylC Control (CEG_SQfC)

The synthetic formylC control was prepared by hybridizing CEG_SQfC_Fwd (100 μM stock) and CEG_SQfC_Rev (100 μM stock) in 1× Anneal buffer (10 mM Tris pH 7.4 and 10 mM NaCl). The oligomers were annealed by heating to 97.5° C. for 2 minutes 30 seconds, then were cooled to 40° C. by decreasing the temperature of 0.1° C. per second, and were then held at 40° C. for 15 minutes.

After library preparation, the DNA was then quantified by Qubit HS dsDNA assay kit.

Step 4: Library Preparation

The sheared human genomic DNA (800 bp, 4.4 μg) was spiked in with 2.5% synthetic formylC control and 10% PCR formylC control. Libraries were prepared using the NEBNext DNA Library Prep Master Mix Set for Illumina (NEB) following the manufacturer's specifications, and using 10 μL of the methylated adapter pair (25 μM) during the adapter ligation step. The methylated adapter pair was prepared by annealing Oligo 9 (100 μM stock) and Oligo 10 (100 μM stock) to a final concentration of 25 μM each in 1× Anneal buffer (10 mM Tris pH 7.4 and 10 mM NaCl). The oligomers were hybridized by heating to 95° C. for 3 minutes, then were cooled to 14° C. by decreasing the temperature of 0.1° C. per second.

After library preparation, the DNA was then quantified by Qubit HS dsDNA assay kit.

Step 5: Denaturing Step

500 ng of the control-spiked human genomic DNA from step 4 in a total volume of 23 μL in ultra pure water was denatured in 1 μL of sodium hydroxide 1 M solution at 37° C. for 30 minutes. 10 identical replicates were prepared and denatured at this step.

Step 6: Reduction

1 μL of the alkaline borohydride solution prepared in step 1 was added to the 24 μL denatured DNA for 8 replicates from step 5. 1 μL of ultra pure water was added to the last 2 replicates (Table 8). Each reaction was incubated at room temperature in the dark for 60 minutes.

TABLE 8
Reaction conditions

| | Index | Denatured | Reductant | | Final |
|---|---|---|---|---|---|
| | (6N) | DNA from step | Vol | H₂O Vol | [Reductant] |
| Sample ID | (step 8) | 5 (μL) | (μL) [stock] | (μL) | [mM] |
| CEG11_139_167b2 | 11 | 24 | 1 [1.27 mM] | — | 50 |
| CEG11_139_168b2 | 12 | 24 | 1 [1.27 mM] | — | 50 |
| CEG11_139_169b2 | 13 | 24 | 1 [1.27 mM] | — | 50 |
| CEG11_139_170b2 | 14 | 24 | 1 [1.27 mM] | — | 50 |
| CEG11_139_171b2 | 15 | 24 | 1 [1.27 mM] | — | 50 |
| CEG11_139_172b2 | 16 | 24 | 1 [1.27 mM] | — | 50 |
| CEG11_139_173b2 | 18 | 24 | 1 [1.27 mM] | — | 50 |
| CEG11_139_174b2 | 19 | 24 | 1 [1.27 mM] | — | 50 |
| CEG11_139_175b2 | 20 | 24 | — | 1 | 50 |
| CEG11_139_176b2 | 21 | 24 | — | 1 | 50 |

Step 7: Bisulfite Conversion

The reduced control-spiked human genomic DNA was bisulfite converted using the TrueMethyl conversion kit (CEGX) following the manufacturers specification. The DNA was then quantified by Qubit ssDNA assay kit.

Step 8: PCR Amplification

PCR amplification was performed on an Agilent Surecycler 8800 thermocycler using 1 μL of the bisulfite converted DNA and 5 U of VeraSeq Ultra DNA polymerase (Enzymatics). Thermocycling conditions consisted of an initial denaturation step at 95° C. for 2 minutes, followed by 15 cycles of:

Denaturation at 95° C. for 30 sec

Annealing at 60° C. for 30 sec

Extension at 72° C. for 1 min 30 sec

And a final extension step at 72° C. for 5 min

The primers that were used are PCR_Uni_Fwd and PCR_IDX_Rev (Table 1). The latter primer includes a sequence that hybridizes to an Illumina flow cell and contains a specific index tag (represented by a string of 6N nucleotides) (Table 3). PCR products were purified as described in step 7.

The products obtained were purified with 2× 18% PEG Ampure XP Beads (18% PEG-8000, 1 M NaCl, 1 mM EDTA, 10 mM Tris pH 8) according to manufacturers instructions but using 80:20 freshly prepared acetonitrile:water instead of 80:20 ethanol:water.

Samples were eluted from the beads in 15 μl ultra pure water. Both controls were then checked on a bioanalyzer and quantified by Qubit HS dsDNA assay kit and by qPCR using the Illumina library Quantification kit (KAPA Biosystems).

Step 9. Sequencing and Analysis:

Sequencing was carried out on an Illumina Miseq sequencer with a paired end run (R1 110 bp and R2 40 bp long). The 10 libraries were pooled at 2 nM and then diluted to 20 pM before loading on to the flow cell and sequenced, according to the manufacturers instructions. The raw output fastq read sequences were quality filtered and trimmed to remove the adapter sequences with the software Trim Galore. The data was aligned to the PCR formylC control and to the synthetic SQfC control with Bismark software and visualized by SeqMonk. Table 9 shows the conversion rates from the sequencing data obtained from a library spiked in with 2.5% of synthetic formylC control and 10% PCR formylC control for 8 repeats using sodium borohydride solution and 2 replicates with no reductant. FIG. 5 shows the average of the reproducibility.

TABLE 9
Repeatability of reduction using the
alkaline sodium borohydride solution

| | | synthetic formylC | PCR formylC |
|---|---|---|---|
| | | control | control |
| Sample | Treatment | fC2U % | fC2U % |
| CEG11_139_167b2 | RedBS | 87.4 | 90.4 |
| CEG11_139_168b2 | RedBS | 87.0 | 90.3 |
| CEG11_139_169b2 | RedBS | 86.7 | 89.5 |
| CEG11_139_170b2 | RedBS | 87.4 | 90.4 |
| CEG11_139_171b2 | RedBS | 87.1 | 90.1 |
| CEG11_139_172b2 | RedBS | 87.5 | 89.3 |
| CEG11_139_173b2 | RedBS | 85.4 | 87.9 |
| CEG11_139_174b2 | RedBS | 86.2 | 88.7 |
| CEG11_139_175b2 | BS | 20.1 | 1.0 |
| CEG11_139_176b2 | BS | 20.0 | 1.0 |

TABLE 10

| | | | Oxidation | Reduction |
|---|---|---|---|---|
| | | | then | then |
| | Regular | Bisulfite | Bisulfite | Bisulfite |
| Base | Sequencing | Sequencing | Sequencing | Sequencing |
| C | C | U | U | U |
| 5mC | C | C | C | C |
| 5hmC | C | C | U | C |
| 5fC | C | U | U | C |

TABLE 11

| | |
|---|---|
| a) | 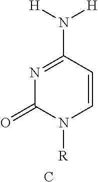 |
| b) | 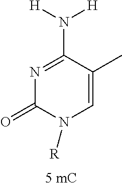 |
| c) | 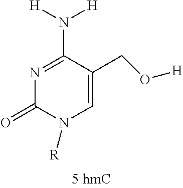 |
| d) | 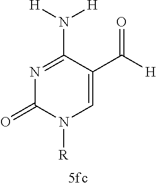 |

### REFERENCES

1. A. M. Deaton et al Genes Dev. 25, 1010 (May 15, 2011).

2. M. Tahiliani et al. Science 324, 930 (May 15, 2009).

3. S. Ito et al. Nature 466, 1129 (Aug. 26, 2010).

4. A. Szwagierczak et al Nucleic Acids Res, (Aug. 4, 2010).

5. K. P. Koh et al. Cell Stem Cell 8, 200 (Feb. 4, 2011).

6. G. Ficz et al., Nature 473, 398 (May 19, 2011).

7. K. Williams et al. Nature 473, 343 (May 19, 2011).

8. W. A. Pastor et al. Nature 473, 394 (May 19, 2011).

9. Y. Xu et al. Mol. Cell 42, 451 (May 20, 2011).

10. M. R. Branco et al Nat. Rev. Genet. 13, 7 (January 2012).

11. S. Kriaucionis et al Science 324, 929 (May 15, 2009).

12. M. Munzel et al. Angew. Chem. Int. Ed. 49, 5375 (July 2010).

13. H. Wu et al. Genes Dev. 25, 679 (Apr. 1, 2011).

14. S. G. Jin et al Nuc. Acids. Res. 39, 5015 (July 2011).

15. C. X. Song et al. Nat. Biotechnol. 29, 68 (January 2011).

16. M. Frommer et al. PNAS. U.S.A. 89, 1827 (March 1992).

17. Y. Huang et al. PLoS One 5, e8888 (2010).

18. C. Nestor et al. Biotechniques 48, 317 (April 2010).

19. C. X. Song et al. Nat. Methods, (Nov. 20, 2011).

20. J. Eid et al. Science 323, 133 (Jan. 2, 2009).

21. E. V. Wallace et al. Chem. Comm. 46, 8195 (Nov. 21, 2010).

22. M. Wanunu et al. J. Am. Chem. Soc., (Dec. 14, 2010).

23. WO2013/017853

24. M. J. Booth et al. Science (2012) 336, 934-937

25. M. J. Booth et al. Nature Protocols (2013) 8, 10, 1841-1851.

37. Li et al Nucleic Acids (2011) Article ID 870726

38. Pfaffeneder, T. et al (2011) Angewandte. 50. 1-6

39. Lister, R. et al (2008) Cell. 133. 523-536

40. Wang et al (1980) Nucleic Acids Research. 8 (20), 4777-4790

41. Hayatsu et al (2004) Nucleic Acids Symposium Series No. 48 (1), 261-262

42. Lister et al (2009) Nature. 462. 315-22

43. Sanger, F. et al PNAS USA, 1977, 74, 5463

44. Bentley et al Nature, 456, 53-59 (2008)

45. K J McKernan et al Genome Res. (2009) 19: 1527-1541

46. M Ronaghi et al Science (1998) 281 5375 363-365

47. Eid et al Science (2009) 323 5910 133-138

48. Korlach et al Methods in Enzymology 472 (2010) 431-455)

49. Rothberg et al (2011) Nature 475 348-352).

1. A method of identifying a 5-formylcytosine residue in a sample nucleotide sequence comprising;
(i) providing a population of single stranded polynucleotides which comprise the sample nucleotide sequence,
(ii) reducing a first portion of said population by adding an alkaline borohydride solution,
(iii) treating the reduced first portion of said population and a second portion of said population with bisulfite,
(iv) sequencing the polynucleotides in the first and second portions of the population following steps ii) and iii) to produce first and second nucleotide sequences, respectively and;
(v) identifying the residues in the first and second nucleotide sequences which correspond to a 5-formylcytosine residue in the sample nucleotide sequence.

2. The method according to claim 1 wherein identification of cytosine at a position in the first nucleotide sequence and uracil at the same position in the second nucleotide sequence is indicative that the cytosine residue in the sample nucleotide sequence is 5-formylcytosine (5fC).

3. The method according to claim 1 comprising;
(i) providing a population of single stranded polynucleotides which comprise the sample nucleotide sequence,
(ii) reducing a first portion of said population by adding an alkaline borohydride solution,
(iii) oxidising a second portion of said population,
(iv) treating the reduced first portion, oxidised second portion and a third portion of said population with bisulfite,
(v) sequencing the polynucleotides in the first, second and third portions of the population following steps ii), iii) and iv) to produce first, second and third nucleotide sequences, respectively and;
(vi) identifying the residues in the first, second and third nucleotide sequences which correspond to a cytosine residue in the sample nucleotide sequence.

4. The method according to claim 3 wherein identification of cytosine at a position in the first, second and third nucleotide sequences is indicative that the cytosine residue in the sample nucleotide sequence is 5-methylcytosine.

5. The method according to claim 1 wherein the first portion of said population is reduced using a solution of alkaline NaBH₄.

6. The method according to claim 1 wherein identification of uracil at a position in both the first and the second nucleotide sequence is indicative that the cytosine residue in the sample nucleotide sequence is unmodified cytosine.

7. The method according to claim 1 comprising;
providing a fourth portion of the population of polynucleotides comprising sample nucleotide sequence; and,
sequencing the polynucleotides in the fourth portion to produce the sample nucleotide sequence.

8. The method according to claim 1 wherein the polynucleotides are genomic DNA.

9. The method according to claim 1 wherein the single stranded polynucleotides are in alkaline solution prior to borohydride treatment.

10. The method according to claim 1 wherein the population of polynucleotides or one or more of the first, second, third and fourth portions of the population are immobilised.

11. The method according to claim 1 wherein one or more of the first, second, third and fourth portions of the population are amplified before sequencing.

12. The method according to claim 11 wherein one or more of the first, second, third portions of the population are amplified following treatment with bisulfite.

13. The method according to claim 1 wherein the final borohydride concentration in step (ii) is 10 to 200 mM.

14. The kit for use in a method of identifying a 5-formylcytosine residue according to claims 1 comprising;
(i) an alkaline borohydride solution; and,
(ii) a bisulfite reagent.

15. The kit according to claim 14 further comprising an alkaline solution.

16. The kit according to claim 14 wherein the alkaline borohydride solution is sodium borohydride at pH greater than 10.0.

17. The kit according to claim 14 wherein the alkaline borohydride solution contains hydroxide.

18. The kit according to claim 17 wherein the hydroxide is present at a concentration of greater than 1 Moles/L.

19. The kit according to claim 17 wherein the hydroxide is present at a concentration of greater than 5 Moles/L.
